(19) United States
(12) Patent Application Publication
(10) Pub. No.: US 2017/0169563 A1
(43) Pub. Date: Jun. 15, 2017

(54) Low-Rank and Sparse Matrix Decomposition Based on Schatten p=1/2 and L1/2 Regularizations for Separation of Background and Dynamic Components for Dynamic MRI

(71) Applicant: Macau University of Science and Technology, Macau (MO)

(72) Inventors: Yong LIANG, Macau (MO); Liang-Yong XIA, Macau (MO); Xu-Xin LIN, Macau (MO); Xiao-Ying LIU, Macau (MO); Kuok-Fan CHAN, Macau (MO)

(21) Appl. No.: 14/965,918

(22) Filed: Dec. 11, 2015

(51) Int. Cl. G06T 7/00; A61B 5/055; G06K 9/52; H04N 19/85; G06T 11/00; G06K 9/62

(52) U.S. Cl. CPC G06T 7/0012; G06T 11/008; G06K 9/6215; G06K 9/52; G06K 9/6267; G06T 7/0079; H04N 19/85; A61B 5/055; G06T 2207/10088; G06T 2207/20048; G06T 2207/20144

(57) ABSTRACT

A method for determining a background component and a dynamic component of an image frame from an under-sampled data sequence obtained in a dynamic MRI application is provided. The two components are determined by optimizing a low-rank component and a sparse component of the image frame in a sense of minimizing a weighted sum of terms. The terms include a Schatten_{p=1/2} (S_{1/2}-norm) of the low-rank component, an L_{1/2}-norm of the sparse component additionally sparsified by a sparsifying transform, and an L₂-norm of a difference between the sensed data sequence and a reconstructed data sequence. The reconstructed one is obtained by sub-sampling the image frame according to an encoding or acquiring operation. The background and dynamic components are the low-rank and sparse components, respectively. Experimental results demonstrate that the method outperforms an existing technique that minimizes a nuclear-norm of the low-rank component and an L₁-norm of the sparse component.

### BACKGROUND

### FIELD OF THE INVENTION

The present invention relates to determining a background component and a dynamic component of an image frame from a sensed data sequence obtained in a dynamic magnetic resonance imaging (MRI) application, where the sensed data sequence is under-sampled with respect to the image frame.

### LIST OF REFERENCES

There follows a list of references that are occasionally cited in the specification. Each of the disclosures of these references is incorporated by reference herein in its entirety.
[1] Lustig, M., Donoho, D., Pauly, J. M., “Sparse MRI: The application of compressed sensing for rapid MR imaging,” Magnetic Resonance in Medicine, 2007, 58(6): pp. 1182-1195.
[2] McGibney, G., et al., “Quantitative evaluation of several partial Fourier reconstruction algorithms used in MRI,” Magnetic Resonance in Medicine, 1993, 30(1): pp. 51-59.
[3] Barger, A. V., et al., “Time resolved contrast enhanced imaging with isotropic resolution and broad coverage using an undersampled 3D projection trajectory,” Magnetic Resonance in Medicine, 2002, 48(2): pp. 297-305.
[4] Mistretta, C. A., et al., “Highly constrained backprojection for time-resolved MRI, Magnetic Resonance in Medicine, 2006, 55(1): pp. 30-40.
[5] Winkelmann, S., et al., “An optimal radial profile order based on the Golden Ratio for time-resolved MRI,” IEEE Transactions on Medical Imaging, 2007, 26(1): pp. 68-76.
[6] Meyer, C. H., Hu, B. S., Nishimura, D. G., and Macovski, A., “Fast spiral coronary artery imaging,” Journal of Magnetic Resonance, 1992, 28: pp. 202-213.
[7] Peters, D. C., et al., “Undersampled projection reconstruction applied to MR angiography,” Journal of Magnetic Resonance, 2000; 43: pp. 91-101.
[8] Sodickson D. K., and Manning, W. J., “Simultaneous acquisition of spatial harmonics (SMASH): fast imaging with radiofrequency coil arrays,” Journal of Magnetic Resonance, 1997, 38: pp. 591-603.
[9] Griswold, M.A., et al., “Generalized autocalibrating partially parallel acquisitions (GRAPPA),” Journal of Magnetic Resonance, 2002, 7: pp. 1202-1210.
[10] Pruessmann, K. P., Weiger, M., Scheidegger, M. B., and Boesiger, P., “SENSE:

sensitivity encoding for fast MRI,” Journal of Magnetic Resonance, 1999, 42: pp. 952-962.
[11] Prieto, C., Mir, R., Batchelor, P. G., Hill, D. L. G., Guarini, M., and Irarrazaval, P., “Reconstruction of under-sampled dynamic frames by modeling the motion of object elements,” Proceedings of the 14th Scientific Meeting of ISMRM, Seattle, 2006, p. 687.
[12] Sharif, B., Derbyshire, J., Faranesh, A., Lederman, R., and Bresler, Y., “Real-time non-gated cardiac MRI using PARADISE: doubly adaptive accelerated imaging,” Proceedings of the 17th Scientific Meeting of ISMRM, Honolulu, 2009: p. 767.
[13] Odille, F., Uribe, S., Batchelor, P. G., Prieto, C, Schaeffter, T., and Atkinson. D., “Model-based reconstruction for cardiac cine MRI without ECG or breath holding,” Journal of Magnetic Resonance, 2010, 63: pp. 1247-1257.
[14] Tsao, J., Boesiger, P., and Pruessmann, K. P., “k-t BLAST and k=SENSE: Dynamic MRI with high frame rate exploiting spatiotemporal correlations,” Magnetic Resonance in Medicine, 2003, 50(5): pp. 1031-1042.
[15] Vasanawala, S. S., Alley, M. T., Hargreaves, B. A., Barth, R. A., Pauly, J. M., and Lustig, M., “Improved pediatric MR imaging with compressed sensing,” Radiology, 2010, 256: pp. 607-616.
[16] Otazo, R., Candes, E., and Sodickson, D. K., “Low-rank plus sparse matrix decomposition for accelerated dynamic MRI with separation of background and dynamic components,” Magnetic Resonance in Medicine, 2014.
[17] Gamper, U., Boesiger, P., and Kozerke, S., “Compressed sensing in dynamic MRI,” Magnetic Resonance in Medicine, 2008, 59: pp. 365-373.
[18] Candes, E. J., and Recht, B., “Exact matrix completion via convex optimization,” Foundations of Computational Mathematics, 2009, 9(6): pp. 717-772.
[19]Lingala, S., Hu, Y., Dibella, E., and Jacob, M., “Accelerated dynamic MRI exploiting sparsity and low-rank structure:—SLR,” IEEE Transactions on Medical Imaging, 2011, 30(5): pp. 1042-1054.
[20] Candes, E., Li, X., Ma, Y., and Wright, J., “Robust principal component analysis?” Journal of the ACM, 2011, 58(3): pp. 1-37.
[21]Peng, Y., Ganesh, A., Wright, J., Xu, W., and Ma, Y., “RASL: Robust alignment by sparse and low-rank decomposition for linearly correlated frames,” IEEE Transactions on Pattern Analysis and Machine Intelligence, 2012, 34(11): pp. 2233-2246.
[22] Gao, H., Cai, J., Shen, Z., and Zhao, H., “Robust principal component analysis-based four-dimensional computed tomography,” Physics in Medicine and Biology, 2011, 56(11): pp. 3181-3198.
[23] Gao, H., et al., “Compressed sensing using prior rank, intensity and sparsity model (PRISM): Applications in cardiac cine MRI,” Proceedings of the 20th Annual Meeting of ISMRM, Melbourne, 2012, p. 2242.
[24] Wright, J., et al., “Robust principal component analysis: Exact recovery of corrupted low-rank matrices via convex optimization,” Advances in neural information processing systems, 2009: pp. 2080-2088.
[25] Candes, E. J., Romberg, J., and Tao, T., “Robust uncertainty principles: Exact signal reconstruction from highly incomplete frequency information,” IEEE Transactions on Information Theory, 2006, 52: pp. 489-509.
[26] Chandrasekaran, V., et al., Technical Report ArXiv: 0906.2220, 2009.
[27] Donoho, D. L., “Compressed sensing,” IEEE Transactions on Information Theory, 2006, 52(4): pp. 1289-1306.
[28] Xu, Z. B., Zhang, H., Wang, Y., Chang, X. Y., and Liang, Y. “L_{1/2} regularization,” Science China Series F, 2010, 40(3): pp. 1-11.
[29] Rao, G., Peng, Y., Xu, Z. B., “Robust sparse and low-rank components decomposition based on S_{1/2} modeling,” Science China Series F, 2013, 43(6): pp. 733-748.
[30] Boyd, S., et al., “Distributed optimization and statistical learning via the alternating direction method of multipliers,” Foundations and Trends® in Machine Learning, 2011, 3(1): pp. 1-122.
[31] Cai, J.-F., Candes, E., and Shen, Z., “A singular value thresholding algorithm for matrix completion,” SIAM Journal on Optimization, 2010, 20(4):pp. 1956-1982.
[32]Daubechies, I., Defrise, M., and De Mol, C., “An iterative thresholding algorithm for linear inverse problems with a sparsity constraint,” Communications on Pure and Applied Mathematics, 2004, 57: pp. 1413-1457.

### Description of Related Art

In many Magnetic Resonance Imaging (MRI) applications, achieving highly-accelerated dynamic MRI is particularly important for clinical practice, such as cardiac imaging and free-breathing abdominal imaging. To assure that dynamic MRI can be executed rapidly and accurately, one needs to consider two key factors: (a) the speed of MRI data acquisition under normal signal transmission; and (b) the speed of image recovery from MRI data samples.

Since MRI image acquisition speed was found to be limited by physical factors such as gradient amplitude, slew-rate, and physiological constraints like nerve stimulation [1], many research studies have been focused on seeking for new approaches to decrease the amount of data acquired without causing corruption of images. Over the past two decades, there were several methods proposed to improve the image-acquisition speed. McGibney et al. [2] have presented Fourier reconstruction algorithms using Hermitian symmetry to reduce the scan time. Barger et al. [3]-[7] have suggested that non-Cartesian sampling techniques with more efficient k-space transversals can save more time than Cartesian sampling. Some research works have taken advantage of local sensitivity of phased array coil elements in parallel imaging in order to accelerate image acquisition, such as SMASH [8], GRAPPA [9] and SENSE [10]. In dynamic imaging, model-based approaches [11]-[13] and time-frequency techniques [3], [14] that make use of spatiotemporal correlations have been proposed to accelerate image acquisition. However, in comparison with multi-detector computed tomography (CT), existing techniques for dynamic MRI imaging still cannot reduce the scan time to a satisfactory level.

As a recently-introduced technique applied to rapid imaging, compressed sensing (CS) aims at reconstructing a signal and an image from a small subset of k-space that represents an original image. Compared with the traditional Nyquist sampling theorem, CS only needs far fewer samples to recover the signal without loss of image information [1] and can be used to exceed the current rapid acquisition techniques according to an acceleration rate [15]. CS was first presented in the literature of Information Theory and Approximation Theory, taking advantage of the facts that an image is usually sparse in some appropriate basis and that this sparse representation is able to be reconstructed from an under-sampled k-space. A successful application of CS needs to comply with three requirements [1]: (a) there exists a sparse representation for a desired image in a specific transform domain; (b) due to k-space under-sampling, aliasing artifacts should be incoherent in the transform domain; and (c) a non-linear reconstruction is used to enforce sparsity of the image representation and to keep the data acquired consistent. Fortunately, MRI conforms to two key conditions for the application of CS [16]. First, a medical image that is naturally compressible can be easily transformed to a sparse representation by choosing an appropriate transform domain such as those related to wavelets, finite differences (total variation), learned dictionaries and many others. Second, instead of directly acquiring pixel samples, MM scanners naturally acquire data in a spatial frequency domain (k-space), which promotes producing incoherent aliasing artifacts by under-sampling a Cartesian k-space randomly or by using non-Cartesian k-space trajectories. Since a video consisting of multiple frames are generally much more compressible than a single static image, one can take advantage of essential spatiotemporal correlations in dynamic MRI data to generate sparser representation than the case of exploiting spatial correlations only.

CS has been applied in several cardiac MRI applications to reduce scan time by maximizing the sparsity of the image reconstructed in a transform domain subject to data consistency constraints [17]. With the progress of CS research in MRI, research has been started to consider how to extend the idea of CS from signal vectors to matrices, in which missing or corrupted entries are recovered under a low-rank condition [18]. In dynamic MM, Lingala et al. [19] have proposed a model that permits decomposing a data matrix into a superposition of a low-rank component (L) and a sparse component (S) via robust principal component analysis (RPCA), the performance of which is improved over classical PCA with sparse outliers. RPCA has been successfully used in the field of computer vision, such as the separation of dynamic components and a static background from a video sequence [20], alignment of an image with deformations induced by the projection of 3D surfaces onto an imaging plane [21], and reconstruction of an image in 4-dimensional CT with a reduced number of projections [22]. By considering each static image corresponding to each temporal frame as a column of a space-time matrix, the L+S matrix decomposition is very suitable for dynamic imaging, where a low-rank matrix (L) can represent the temporally correlated background and a sparse matrix (S) can represent the dynamic component that lies on top of the background. Gao et al. [23] have applied the L+S model to dynamic MRI in reconstructing under-sampled cardiac cine data sets with separation of cardiac motion from a static background among frames. Recently, as a result that the L+S model can be used to reconstruct an under-sampled k-space with increased compressibility of dynamic MRI data [16], the combination of CS and L+S matrix decomposition brings an attractive improvement to further increase the imaging speed of dynamic MRI.

As the advantages of the L+S matrix decomposition are apparent, it is desirable if the performance in separating the low-rank component and the sparse component can be further improved over existing L+S decomposition techniques, where the aforesaid performance is measured in terms of the rank of the obtained low-rank component and/or the sparsity of the sparse component. There is a need in the art for a novel technique that can achieve an improvement in the separation performance.

### SUMMARY OF THE INVENTION

Hereinafter the Schattenp-norm and the Lebesgue's p-norm are denoted by Sₚ and Lₚ, respectively, unless otherwise stated.

An aspect of the present invention is to provide a method for determining a background component and a dynamic component of an image frame from a sensed data sequence obtained in a dynamic MM application. The sensed data sequence is under-sampled with respect to the image frame.

The method comprises numerically optimizing a low-rank component and a sparse component of the image frame in a sense of minimizing a weighted sum of terms under a condition that the image frame is a sum of the low-rank component and the sparse component. In particular, the terms include a S_{1/2}-norm of the low-rank component, an L_{1/2}-norm of the sparse component additionally sparsified by a sparsifying transform, and an L₂-norm of a difference between the sensed data sequence and a reconstructed data sequence. The reconstructed data sequence is obtained by sub-sampling the image frame according to an encoding or acquiring operation. The background component is the optimized low-rank component, and the dynamic component is the optimized sparse component.

Preferably, the weighted sum of terms follows the one shown in EQN. (5) below.

It is also preferable that the low-rank component and the spare component are numerically optimized by an iterative algorithm adopted from Algorithm 1 to be detailed below.

Other aspects of the present invention are disclosed as illustrated by the embodiments hereinafter.

### BRIEF DESCRIPTION OF THE DRAWINGS

FIG. 1 depicts the experimental results obtained for cardiac perfusion, where the reconstructed image, the low-rank matrix image and the sparse matrix image obtained by L+S model with S₁ and L₁ regularizations are shown on the top, in the middle and at the bottom, respectively.

FIG. 2 depicts the experimental results obtained for cardiac perfusion, where the reconstructed image, the low-rank matrix image and the sparse matrix image obtained by L+S model with S_{1/2} and L_{1/2} regularizations are shown on the top, in the middle and at the bottom, respectively.

FIG. 3 depicts some features respectively extracted from the third frames of FIG. 1 and FIG. 2, where for each of the models, the low-rank and the sparse components are first obtained and then the reconstructed frame is generated.

FIG. 4 depicts the experimental results obtained for cardiac cine data, where the reconstructed image, the low-rank matrix image and the sparse matrix image obtained by L+S model with S₁ and L₁ regularizations are shown on the top, in the middle and at the bottom, respectively.

FIG. 5 depicts the experimental results obtained for cardiac cine data, where the reconstructed image, the low-rank matrix image and the sparse matrix image obtained by L+S model with S_{1/2} and L_{1/2} regularizations are shown on the top, in the middle and at the bottom, respectively.

FIG. 6 depicts some features respectively extracted from the third frames of FIG. 4 and FIG. 5, where for each of the models, the low-rank and the sparse components are first obtained and then the reconstructed frame is generated.

FIG. 7 depicts the experimental results obtained for abdominal data, where the reconstructed image, the low-rank matrix image and the sparse matrix image obtained by L+S model with S₁ and L₁ regularizations are shown on the top, in the middle and at the bottom, respectively.

FIG. 8 depicts the experimental results obtained for abdominal data, where the reconstructed image, the low-rank matrix image and the sparse matrix image obtained by L+S model with S_{1/2} and L_{1/2} regularizations are shown on the top, in the middle and at the bottom, respectively.

FIG. 9 depicts some features respectively extracted from the third frames of FIG. 7 and FIG. 8, where for each of the models, the low-rank and the sparse components are first obtained and then the reconstructed frame is generated.

FIG. 10 depicts a flowchart exemplarily illustrating a method for determining a background component and a dynamic component from an under-sampled sensed data sequence in dynamic MRI.

FIG. 11 depicts an example illustrating a MRI data-analysis system that acquires sensed data from a MRI machine and performs data analysis.

### DETAILED DESCRIPTION

Traditionally, the L+S matrix decomposition has been performed by transforming the decomposition problem into a convex optimization problem of minimizing a nuclear-norm (viz., a sum of singular values) of a low-rank section of the matrix and an L₁-norm (namely, a sum of absolute values) of a sparse section of the matrix subject to data consistency constraints [24]. Different from the traditional approach, herein in the present invention, an S_{1/2}-norm and an L_{1/2}-norm are used to replace the nuclear-norm and the L₁-norm, respectively, for improving the performance of the L+S matrix decomposition.

The technique developed by the aforementioned improved approach was tested in experiments. To guarantee fairness and effectiveness in the experiments, several sets of data of dynamic MRI experiments from [16] were used, where joint multi-coil reconstruction was used for Cartesian and non-Cartesian k-space sampling. As will be shown, experimental results demonstrate the superiority of the disclosed technique by a set of measures in three dynamic MRI with true acceleration including cardiac perfusion, cardiac cine and free-breathing accelerated abdominal dynamic contrast enhanced experiments (DCE)

### A. Development of the Disclosed Technique

### A.1 Low-Rank and Sparse Matrix Decomposition

In a dynamic MRI application, an image frame M can generally be decomposed into a low-rank matrix L and a sparse matrix S, where the low-rank matrix L has few non-zero singular values, and the sparse matrix S has few non-zero entries. The non-zero singular values of L represent the background component of M, and the non-zero entries of S represent the dynamic component of M. Note that M=L+S.

The mathematical model of the decomposition problem can be formulated by finding L and S to have

min(rank(L)+λ∥S∥_{L}_{₀})subject to M=L+S, (1)

where: rank(L) is the rank of L; ∥S∥_{L}_{₀} is the L₀-norm of S, which is used to induce more sparse component; and λ is a parameter used to balance the contribution of the L₀-norm.

Obviously, the low-rank and sparse decomposition usually is a morbid optimization problem. Inspired by compression perception and statistical fields [25]-[27], we can rewrite EQN. (1) as

min(∥L∥*+λ∥S∥_{L}_{₁} subject to M=L+S, (2)

where: ∥L∥* is the nuclear norm (S₁-norm), which is the sum of the singular values of L; ∥S∥_{L}_{₁} is the L₁-norm, which is sum of absolute values of the entries of S; and λ is a parameter used to balance the contribution of the L₁-norm of S.

According to [28] and [29], EQN. (2) can be rewritten as:

min(∥L∥_{S}_{_{1/2}}^{1/2}+λ∥S∥_{L}_{_{1/2}}^{1/2}) subject to M=L+S, (3)

where: ∥L∥_{S}_{_{1/2}}^{1/2} is the S_{1/2}-norm of the low-rank component L, used to induce the lower rank component of the image frame M; ∥S∥_{L}_{_{1/2}}^{1/2} is the L_{1/2}-norm, used to induce more sparse component; and λ is a parameter used to balance the contribution of the L_{1/2}-norm of S.

In [28], Xu et al. have demonstrated many attractive properties of the L_{1/2}-norm penalty, such as unbiasedness, sparsity and oracle properties. In [29], Rao et al. have demonstrated that the S_{1/2}-norm can induce the lower rank components for the components decomposition. It is expected that these features provide a better performance for the decomposition technique that is developed herein.

### A.2 Low-Rank and Sparse Matrix Reconstruction Under-Sampled Dynamic MRI

In case CS is used to obtain dynamic MRI data, the image frame M is required to be reconstructed from an under-sampled sensed data sequence. The sensed data sequence is under-sampled with respect to the image frame.

According to [16], ∥TS∥_{L}_{_{1/2}}^{1/2} instead of ∥S∥_{L}_{_{1/2}}^{1/2} is used, and EQN. (3) is modified to reconstruct the under-sampled dynamic MM data, where T is a sparsifying transform. One example of the sparsifying transform is given in [16]. The mathematical model is reformulated to determine L and S where the determined L and S have

min(∥L∥_{S}_{_{1/2}}^{1/2}+λ∥TS∥_{L}_{_{1/2}}^{1/2}), subject to E(L+S)=d, (4)

in which: E is an encoding or acquisition operator, one example of which is given in [16]; and d is under-sampled k-t space data sequence, which is an acquired time series of the 2D or 3D Fourier transform of frames (k-space) at varying time points t. Note that as indicated in EQN. (4), the data sequence d containing sensed dynamic MM data is related to the image frame M by sub-sampling the image frame M according to the encoding or acquiring operation E.

Since d in practice is usually corrupted and obtained in the presence of noise and interference during measurement, we add a regularization function to EQN. (4) to take into consideration of the corrupted d. It gives

min[1/2∥E(L+S)−d∥_{L}_{₂}²+λ_{L}∥L∥_{S}_{_{1/2}}^{1/2}+λ_{S}∥TS∥_{L}_{_{1/2}}^{1/2}] (5)

where λ_{L} is a singular-value threshold, λ_{S} is a sparsity threshold, and ∥∥_{L}_{₂}² denotes an L₂-norm. The values of λ_{L} and λ_{S} are pre-determined, and reflect a balance in the contributions of the three terms in EQN. (5).

To solve the optimization problem given by EQN. (5), there have been some approaches proposed such as alternating direction [20], split Bregman [14], or other convex optimization techniques. In [16], Otazo et al. have used the iterative soft-thresholding to solve the problem, and Λ_{λ}(X) which represents a soft-thresholding operator [30] or a shrinkage operator, is defined on scalars as

$${Λ}_{λ} ( x ) = \{ \begin{matrix}x - sgn ( x ) \times λ , & \vert x \vert > λ \\ 0 & otherwise\end{matrix} \qquad ( 6 )$$

where x is a component in a matrix.

Preferably, iterative half-thresholding [29] instead of iterative soft-thresholding is used, as in the present work. The half-thresholding or shrinkage operator is defined on scalars as

$$\begin{matrix}{H}_{λ} ( x ) = \{ \begin{matrix}\frac{2}{3} x ( 1 + cos ( \frac{2 π}{3} - \frac{2 ϕ ( x )}{3} ) ) , & \vert x \vert > \frac{\sqrt[3]{54}}{4} {λ}^{2 / 3} \\ 0 & otherwise\end{matrix} \\ where & ( 7 ) \\ ϕ ( x ) = {cos}^{- 1} [ \frac{λ}{8} \cdot {( \frac{\vert x \vert}{3} )}^{- 3 / 2} ] . & ( 8 )\end{matrix}$$

B. Algorithm for the L+S Decomposition Based on S_{1/2} and L_{1/2} Regularizations

Denote U and V such that M=UΣV^{H} is the singular value decomposition of M , where Σ is a matrix containing singular values of M. Then the (dominant) singular values are extracted by computing SVT_{λ}(M)=UH_{λ}(Σ)V^{H} where SVT_{λ}(M) denotes a Singular Value Thresholding (SVT) operator under a selected value of λ.

Details of the developed algorithm of low-rank and sparse reconstruction from under-sampled dynamic MM data are as follows. At the k th iteration, we use Mₖ₋₁−Sₖ₋₁ to apply to the SVT operator and then utilize Mₖ₋₁−Lₖ₋₁ to apply to the shrinkage operator H_{λ}(X). The new Mₖ is obtained by performing data consistency, where E^{H}(E(Lₖ+Sₖ−d)) is subtracted from Lₖ+Sₖ. Algorithm 1 represents a combination of singular value thresholding used for matrix completion [31] and iterative half-thresholding used for sparse reconstruction [32].

Algorithm 1: an algorithm for the L +
S decomposition using iterative half-thresholding

Input:

d : under-sampled k−t data
E : the encoding or acquisition operator
T : the sparsifying transform
λ_{L}, λ_{S} : regularization parameters

Initialization: M₀ = E^{H} d, S₀ = 0, L₀ = 0, k=1;
While not converged, do

Sₖ = T⁻¹ (H_{λ}(T(Mₖ₋₁ − Lₖ₋₁)));
Lₖ = UH_{λ}(Mₖ₋₁ − Sₖ₋₁)V^{H} where U and V are obtained from singular

value decomposition on M₀;

Mₖ = Lₖ+ Sₖ − E^{H} (E(Lₖ + Sₖ) − d);
k = k + 1;

End while

### C. Experimental and Result Analysis

### C.1 Datasets and Computing Devices Used in the Experiments

Three video datasets were used in our experiments for demonstrating the superiority of the disclosed technique. The datasets are related to cardiac perfusion, cardiac cine and abdominal. A brief introduction of three datasets is given in Table 1 below, which lists four properties such as the name of datasets, medical devices, the size of frame and the number of frame in the videos [16].

TABLE 1
A brief introduction of three implementation datasets

| | | | Number of |
|---|---|---|---|
| Name | Medical device | Size of frame | frames |
| cardiac | A modified TurboFLASH; 3T | 128 × 128 | 40 |
| perfusion | scanner using a 12-element | | |
| | matrix coil array | | |
| cardiac | 3T scanner using a 12-element | 256 × 256 | 24 |
| cine | matrix coil array | | |
| Abdominal | A 3D stack-of-stars FLASH; | 384 × 384 | 28 |
| | 3T scanner using a 12-element | | |
| | matrix coil array | | |

All the experiments were performed using MATLAB (R2014b) on a workstation with Intel Core i7-4790 processor of 3.60 GHz and 8 GB of RAM equipped with Windows 8.1 OS.

### C.2 Results for Cardiac Perfusion Data

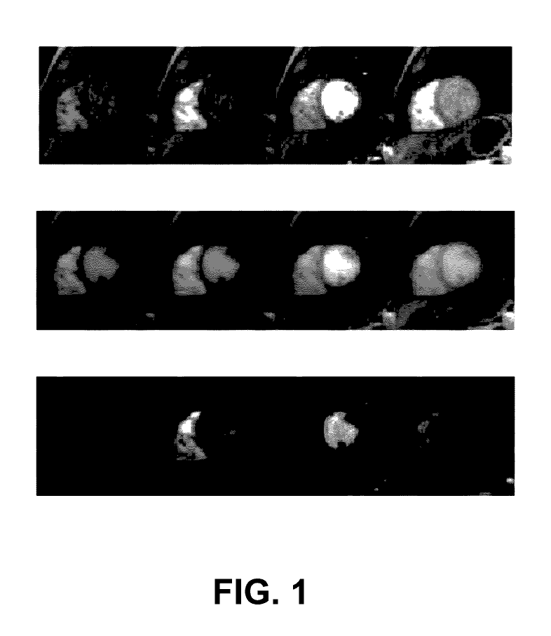

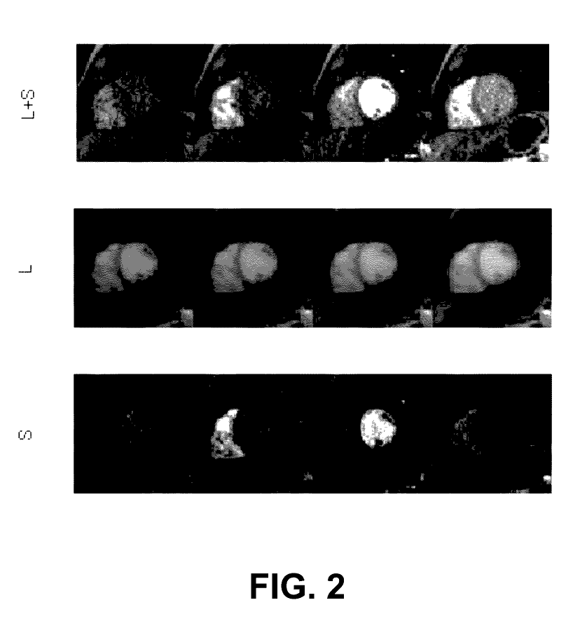

In this section, we evaluate the performance for cardiac perfusion between the two L+S models, the first one with S₁+L₁ regularizations and the second one with S_{1/2}+L_{1/2} regularizations. FIGS. 1 and 2 show the results for the first model and the second model, respectively. In each of FIGS. 1 and 2, the reconstructed dynamic MRI frame, the low-rank matrix image and the sparse matrix image obtained by the respective model are depicted on the top, in the middle, and at the bottom, respectively. As mentioned above, each low-rank frame is a background component and each sparse frame is a dynamic component. It is observed that both the L+S decomposition models could successfully separate the background and the dynamic components of the dynamic MRI data from the video.

We demonstrate as follows that the disclosed technique employing the S_{1/2}+L_{1/2}, approach can induce a lower rank and more sparse components for better reconstructing the dynamic MRI image. Table 2 lists the results of the size of frame, the rank of low-rank component, the number of zeroes and the percentage of the number of zeroes respectively obtained by the S₁+L₁ and the S_{1/2}+L_{1/2} regularizations.

TABLE 2
Comparison between the S₁ + L₁ and the S_{1/2} +
L_{1/2} regularizations for the cardiac perfusion data.

| | | | | Percentage of |
|---|---|---|---|---|
| Name | Method | Rank(L) | Zeroes(S) | Zeroes (S) |
| cardiac | S₁ + L₁ | 5 | 31526 | 19.24% |
| perfusion | S_{1/2} + L_{1/2} | 1 | 46298 | 28.26% |

From Table 2, it is observed that the rank of the low-rank components obtained by the S_{1/2}+L_{1/2} method is 1, which is lower than 5 when compared with the S₁+L₁ method. Furthermore, for all the frames, the percentage of the number of zeroes in the sparse components obtained by the S_{1/2}+L_{1/2} method is 28.26%, which is higher than 19.24% as obtained by the S₁+L₁ method. We also mention that the four frames of the background components obtained by the disclosed method are similar.

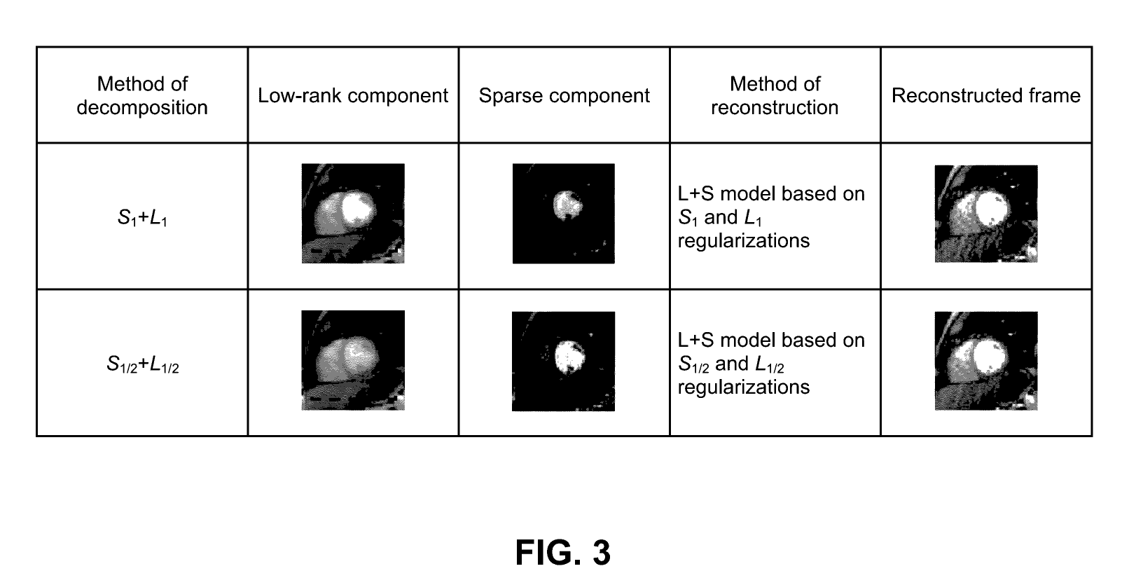

For demonstration, FIG. 3 depicts some features respectively extracted from the third frames of FIG. 1 and FIG. 2. It is apparent that for the background components, the background information is well-kept in the low-rank matrices by both of the L+S decomposition models. However, the rank of the low-rank matrix obtained by the S_{1/2}+L_{1/2} method is lower than that of the S₁+L₁ method. On the other hand, the dynamic components obtained by the disclosed S_{1/2}+L_{1/2} method clearly maintain much more information when compared with the S₁+L₁ method. For example, the image in the dash-line rectangle for the S_{1/2}+L_{1/2} method is brighter and clearer than that of the S₁+L₁ method. The reason we believe is that the non-zero values of the pixels in the spare matrix obtained by the L_{1/2} method is higher than those of the L₁ method, while such pixels which represent the important dynamic information are selected to be more sparse by the L_{1/2} method.

### C. 3 Results for Simulated Cardiac Cine Data

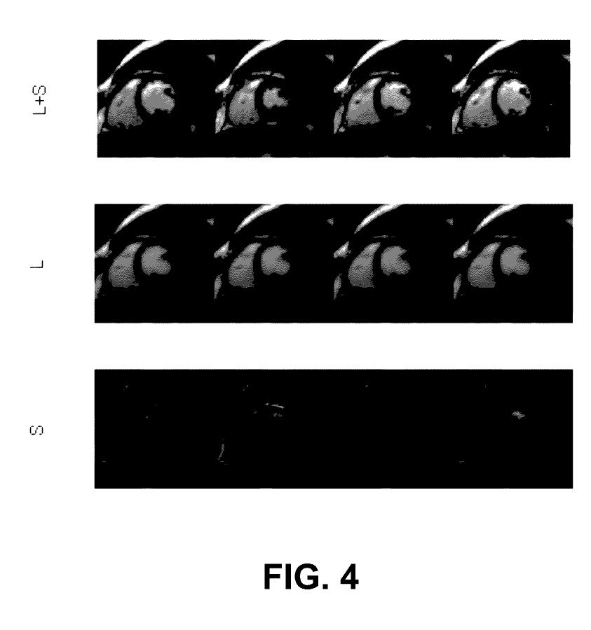

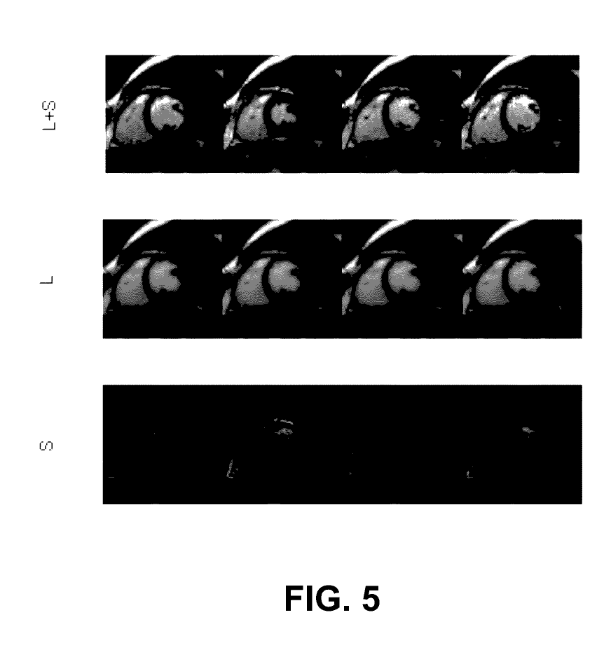

In this section, the results were obtained from the two L+S decomposition methods by using cardiac cine data in the experiments. FIGS. 4 and 5 show the results for the S₁+L₁ method and the S_{1/2}+L_{1/2} method, respectively. In each of FIGS. 4 and 5, the reconstructed dynamic MRI frame, the low-rank matrix image and the sparse matrix image obtained by the respective model are depicted on the top, in the middle, and at the bottom, respectively.

Table 3 lists the results of the size of frame, the rank of low-rank component, the number of zeroes and the percentage of the number of zeroes respectively obtained by the S₁+L₁ and the S_{1/2}+L_{1/2} regularizations.

TABLE 3
Comparison between the S₁ + L₁ and the S_{1/2} +
L_{1/2} regularizations for the cardiac cine data.

| | | | | Percentage of |
|---|---|---|---|---|
| Name | Method | Rank(L) | Zeroes(S) | Zeroes (S) |
| cardiac cine | S₁ + L₁ | 1 | 70738 | 17.99% |
| | S_{1/2} + L_{1/2} | 1 | 107313 | 27.30% |

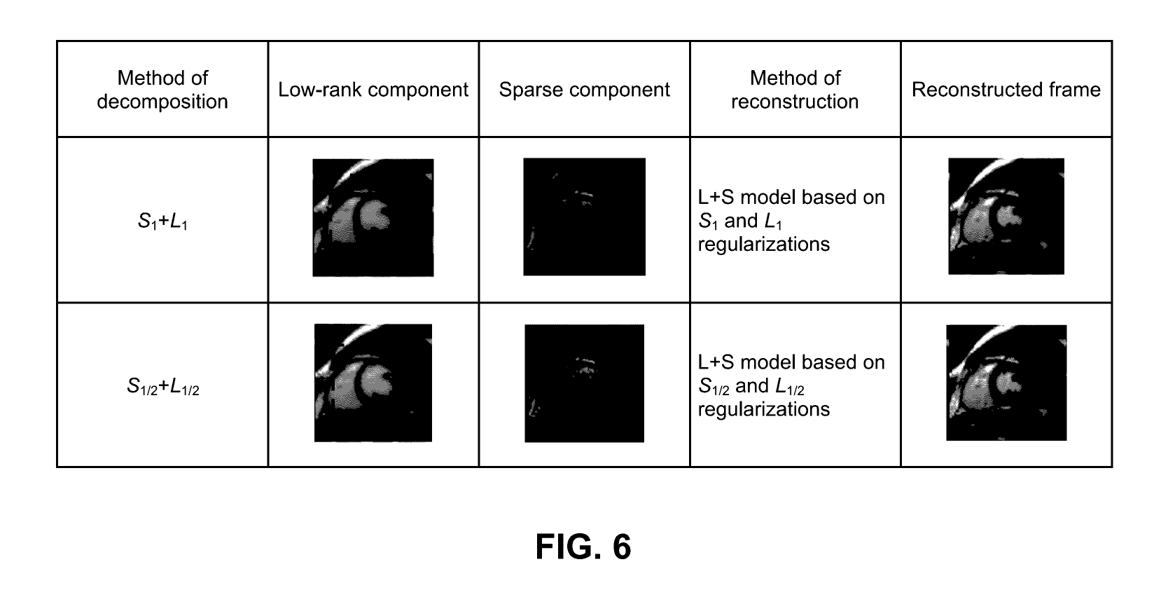

It is apparent from Table 3 that the S_{1/2}+L_{1/2} method as disclosed herein has a capability to induce sparser components than the S₁+L₁ method. For example, for all the frames, the percentage of the number of zeroes in the sparse components obtained by the S_{1/2}+L_{1/2} method is 27.3%, which is higher than 17.99% as obtained by the S₁+L₁ method. In addition, the rank of the low-rank components obtained by the S_{1/2}+L_{1/2} method is the same as that of the S₁+L₁ method. FIG. 6 depicts some features respectively extracted from the third frames of FIG. 4 and FIG. 5. From FIG. 6, it is apparent that the enclosed area in dash line indicates that the disclosed decomposition method is more effective than the S₁+L₁ method for keeping the dynamic information in the dynamic components.

### C. 4 Results for Simulated Abdominal DCE Data

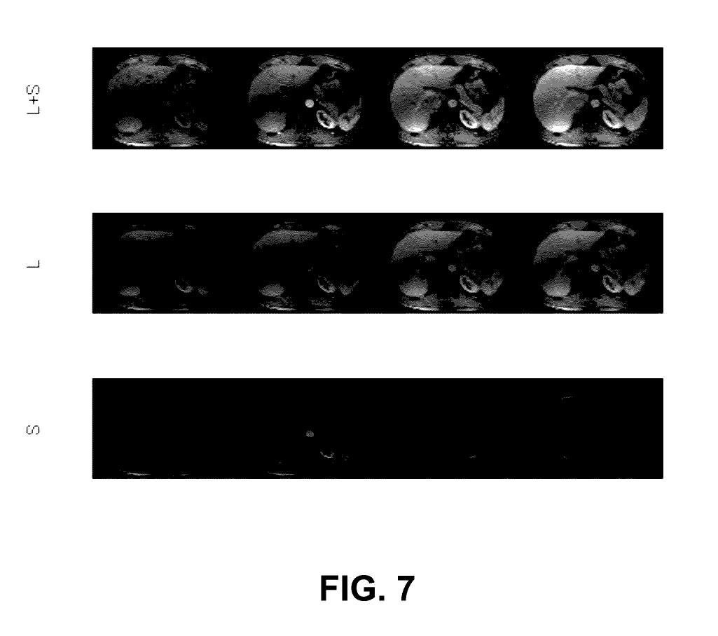

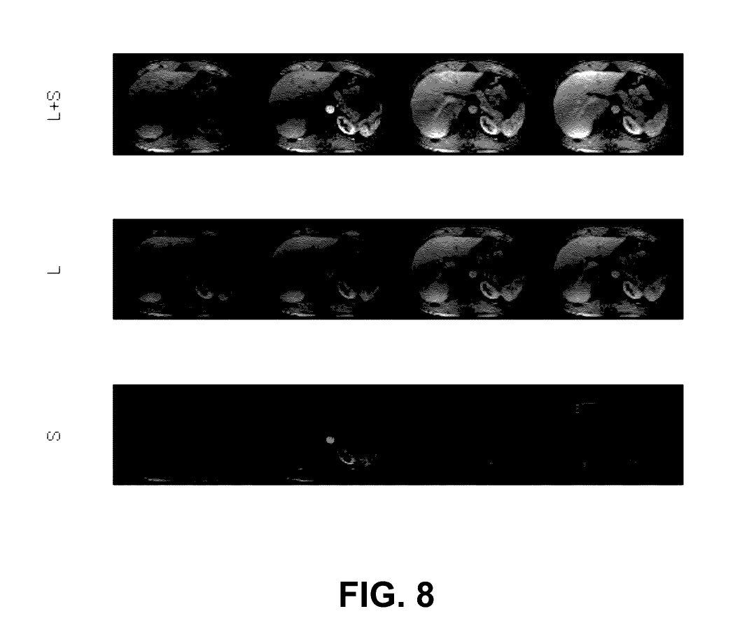

In this section, the results were obtained from the two L+S decomposition methods by using abdominal DCE data in the experiments. FIGS. 7 and 8 show the results for the S₁+L₁ method and the S_{1/2}+L_{1/2} method, respectively. In each of FIGS. 7 and 8, the reconstructed dynamic MRI frame, the low-rank matrix image and the sparse matrix image obtained by the respective model are depicted on the top, in the middle, and at the bottom, respectively.

Table 4 lists the results of the size of frame, the rank of low-rank component, the number of zeroes and the percentage of the number of zeroes respectively obtained by the S₁+L₁ and the S_{1/2}+L_{1/2} regularizations.

TABLE 4
Comparison between the S₁ + L₁ and the S_{1/2} +
L_{1/2} regularizations for the abdominal data.

| | | | | Percentage of |
|---|---|---|---|---|
| Name | Method | Rank(L) | Zeroes(S) | Zeroes (S) |
| abdominal | S₁ + L₁ | 1 | 506999 | 17.34% |
| | S_{1/2} + L_{1/2} | 1 | 523160 | 17.89% |

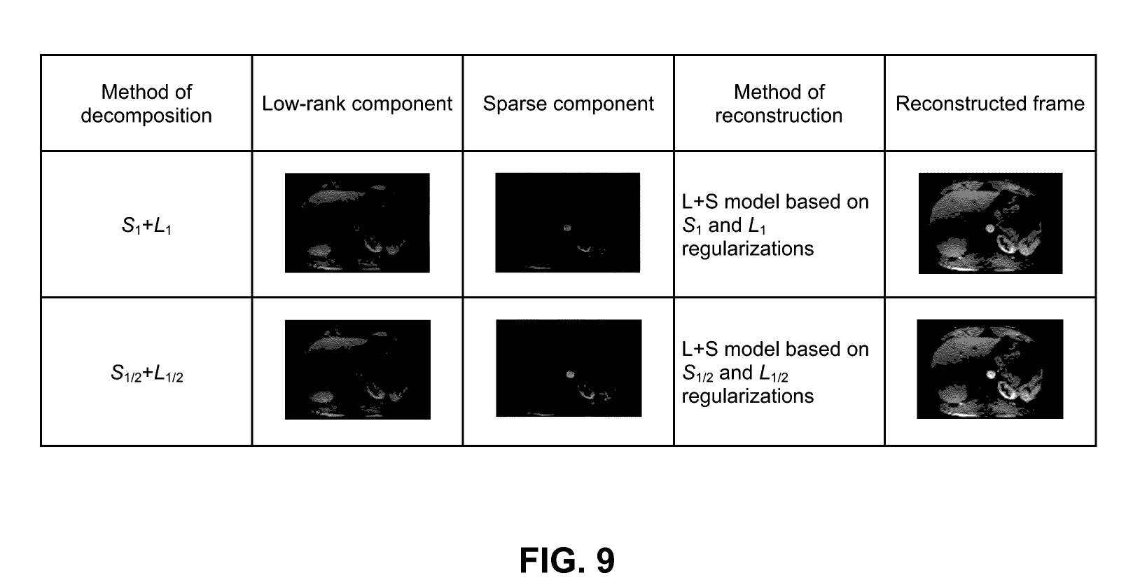

It is apparent from Table 4 that the S_{1/2}+L_{1/2} method as disclosed herein has a capability to induce sparser components than the S₁+L₁ method. For example, for all the frames, the percentage of the number of zeroes in the sparse components obtained by the S_{1/2}+L_{1/2} method is 17.89%, which is higher than 17.34% as obtained by the S₁+L₁ method. In addition, the rank of the low-rank components obtained by the S_{1/2}+L_{1/2} method is the same as that of the S₁+L₁ method. FIG. 9 depicts some features respectively extracted from the third frames of FIG. 7 and FIG. 8. From FIG. 9, it is apparent that the enclosed area in dash line indicates that the disclosed decomposition method is more effective than the S₁+L₁ method for keeping the dynamic information in the dynamic components.

### D. The Present Invention

An aspect of the present invention is to provide a method for determining a background component and a dynamic component of an image frame from a sensed data sequence obtained in a dynamic MRI application. The sensed data sequence is under-sampled with respect to the image frame. The method is realizable by one or more computing devices for dynamic MM applications. Examples of any one computing device include a processor, a computer, a computing server, and a distributed server implemented in a computing cloud.

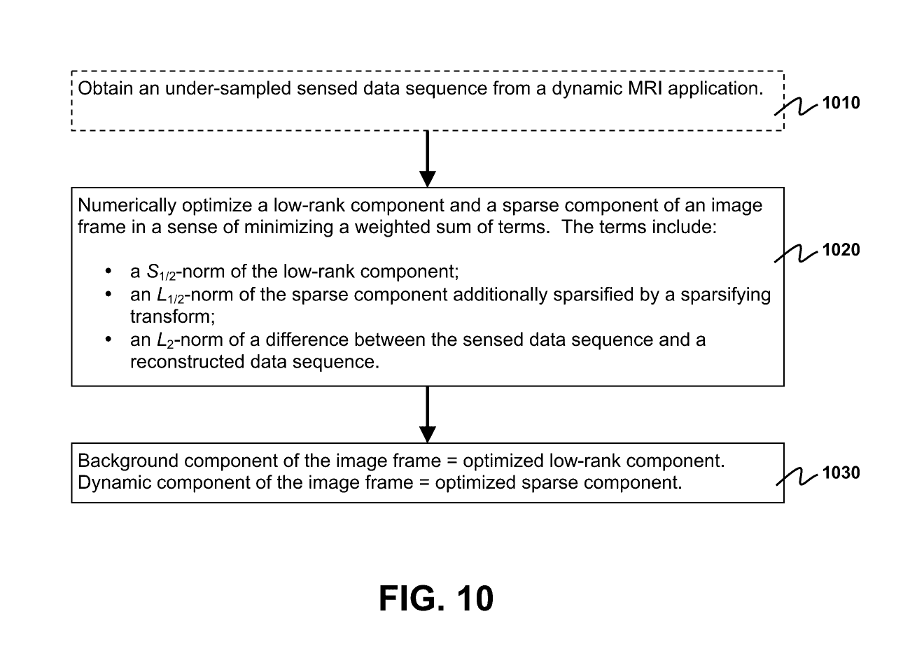

The method is illustrated with the aid of FIG. 10, which depicts a flowchart showing the steps of this method in accordance with an exemplarily embodiment of the present invention. First, the under-sampled sensed data sequence is made available, e.g., by obtaining from the dynamic MM application (step 1010). The sensed data sequence is used to reconstruct the image frame as well as a low-rank component and a sparse component of the image frame. These two components are determined from the sensed data sequence by numerically optimizing the low-rank component and the sparse component in a sense of minimizing a weighted sum of terms, as is shown in EQN. (5) as one example, under a condition that the image frame is a sum of the low-rank component and the sparse component (step 1020). As is also reflected in EQN. (5), the terms include a S_{1/2}-norm of the low-rank component, an L_{1/2}-norm of the sparse component additionally sparsified by a sparsifying transform, and an L₂-norm of a difference between the sensed data sequence and a reconstructed data sequence. The reconstructed data sequence is obtained by sub-sampling the image frame according to an encoding or acquiring operation. As is mentioned above, the background component is the optimized low-rank component and the dynamic component is the optimized sparse component (step 1030).

Preferably, the weighted sum of terms considered in numerical optimization follows the one shown in EQN. (5), and is given as +½∥E(L+S)−d ₂²+λ_{L}·∥L∥_{S}_{_{1/2}}^{1/2}+λ_{S}·∥TS∥_{L}_{_{1/2}}^{1/2}.

It is also preferable that the low-rank component and the spare component are numerically optimized by an iterative algorithm adopted from Algorithm 1 detailed above. In particular, the iterative algorithm comprises a step of iteratively computing Mₖ, Lₖ and Sₖ from Mₖ₋₁, Lₖ₋₁, and Sₖ₋₁, k being a positive integer, with M₀=E^{H}d and S₀=0 until both computed sequences {Lₖ} and {Sₖ} converge. In this step, Mₖ, Lₖ and Sₖ are computed by Sₖ=T⁻¹H_{λ}(T(Mₖ₋₁−Lₖ₋₁)), Lₖ=UH_{λ}(Mₖ₋₁−Sₖ₋₁)V^{H} and Mₖ=Lₖ+Sₖ−E^{H}(E(Lₖ+Sₖ), where H_{λ}(x) is given by EQN. (7).

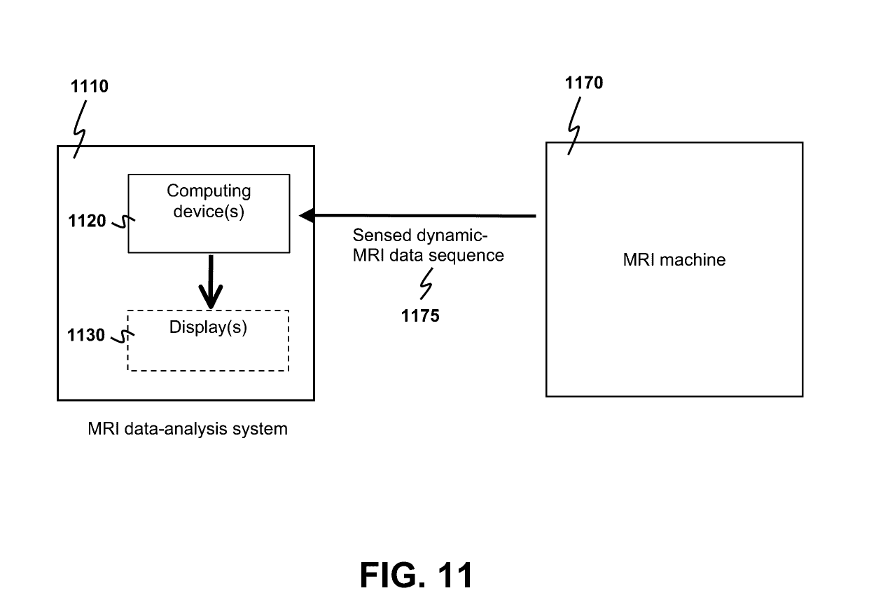

Any embodiment of the disclosed method is implementable in a MRI data-analysis system for analyzing dynamic-MRI sensed data. For illustration, FIG. 11 depicts a case of a MRI data-analysis system 1110 configured to receive a sensed dynamic-MRI data sequence 1175 from a MRI machine 1170 for performing MRI on a human patient or any living subject. The MRI data-analysis system 1110 comprises one or more computing devices 1120 for analyzing the sensed data sequence 1175. As examples, the one or more computing devices 1120 can be processor(s), computer(s), computing server(s), and distributed server(s) implemented in a computing cloud. In particular, the one or more computing devices 1120 are configured to execute a process for determining a background component and a dynamic component of an image frame from the sensed data sequence 1175 according to one of the embodiments of the method disclosed above. The background component and the sparse component as determined may be further processed to yield another result useful for, e.g., medical diagnosis. This result may be presented at one or more displays 1130 of the MRI data-analysis system 1110 for visualization.

The embodiments disclosed herein may be implemented using general purpose or specialized computing devices, computer processors, or electronic circuitries including but not limited to digital signal processors, application specific integrated circuits, field programmable gate arrays, and other programmable logic devices configured or programmed according to the teachings of the present disclosure. Computer instructions or software codes running in the general purpose or specialized computing devices, computer processors, or programmable logic devices can readily be prepared by practitioners skilled in the software or electronic art based on the teachings of the present disclosure.

The present invention may be embodied in other specific forms without departing from the spirit or essential characteristics thereof. The present embodiment is therefore to be considered in all respects as illustrative and not restrictive. The scope of the invention is indicated by the appended claims rather than by the foregoing description, and all changes that come within the meaning and range of equivalency of the claims are therefore intended to be embraced therein.

What is claimed is:

1. A method for determining, by one or more computing devices, a background component and a dynamic component of an image frame from a sensed data sequence obtained in a dynamic magnetic resonance imaging (MRI) application, the sensed data sequence being under-sampled with respect to the image frame, the method comprising:
numerically optimizing a low-rank component and a sparse component of the image frame in a sense of minimizing a weighted sum of terms under a condition that the image frame is a sum of the low-rank component and the sparse component, wherein the terms include:
a Schatten_{p-1/2}-norm (S_{1/2}-norm) of the low-rank component;
an L_{1/2}-norm of the sparse component additionally sparsified by a sparsifying transform; and
an L₂-norm of a difference between the sensed data sequence and a reconstructed data sequence, the reconstructed data sequence being obtained by sub-sampling the image frame according to an encoding or acquiring operation;
whereby the background component is the optimized low-rank component and the dynamic component is the optimized sparse component.

2. The method of claim 1, wherein the weighted sum of terms is given by
½∥E(L+S)−d∥_{L}_{₂}²+λ_{L}∥L∥_{S}_{_{1/2}}^{1/2}+λ_{S}∥TS∥_{L}_{_{1/2}}^{1/2}
where:
L is the low-rank component;
S is the sparse component;
d is the sensed data sequence;
λ_{L} is a pre-determined singular-value threshold;
λ_{S} is a pre-determined sparsity threshold;
T denotes the sparsifying transform;
E denotes the encoding or acquiring operation;
∥∥_{S}_{_{1/2}}^{1/2} denotes a S_{1/2}-norm;
∥∥_{L}_{_{1/2}}^{1/2} denotes an L_{1/2}-norm; and
∥∥_{L}_{₂}² denotes an L₂-norm.

3. The method of claim 2, wherein the low-rank component and the spare component are numerically optimized by an iterative algorithm comprising:
iteratively computing Mₖ, Lₖ and Sₖ from Mₖ₋₁, Lₖ₋₁ and Sₖ₋₁, k a positive integer, with M₀=E^{H}d and S₀=0 until both computed sequences {Lₖ{ and {Sₖ} converge, wherein Sₖ=T⁻¹H_{λ}(T(Mₖ₋₁−Lₖ₋₁)), Lₖ=UH_{λ}(Mₖ₋₁−Sₖ₋₁)V^{H} and Mₖ=Lₖ+Sₖ−E^{H}(E(Lₖ+Sₖ)−d), whereby the optimized low-rank component is the lastly obtained Lₖ and the optimized sparse component is the lastly obtained Sₖ;
where:
Mₖ is the image frame computed at a k th iteration;
Lₖ is the low-rank component computed at the k th iteration;
Sₖ is the sparse component computed at the k th iteration;
U and V are obtained by a singular value decomposition of M₀ such that M₀=UΣV^{H} , Σ containing singular values of M₀; and
H_{λ}(x) is a half-thresholding or shrinking operator defined on scalars as
$${H}_{λ} ( x ) = \{ \begin{matrix}\frac{2}{3} x ( 1 + cos ( \frac{2 π}{3} - \frac{2 ϕ ( x )}{3} ) ) , & \vert x \vert > \frac{\sqrt[3]{54}}{4} {λ}^{2 / 3} \\ 0 & otherwise\end{matrix}$$
in which
$$ϕ ( x ) = {cos}^{- 1} [ \frac{λ}{8} \cdot {( \frac{\vert x \vert}{3} )}^{- 3 / 2} ] .$$

4. The method of claim 1, wherein the low-rank component and the sparse component are numerically optimized by a convex optimization technique.

5. The method of claim 4 wherein the convex optimization technique is an alternating direction technique, a split Bregman technique, or an iterative thresholding technique.

6. The method of claim 1, wherein the low-rank component and the sparse component are numerically optimized by an iterative soft-thresholding technique.

7. The method of claim 1 wherein the low-rank component and the sparse component are numerically optimized by a half-thresholding technique using a half-thresholding or shrinking operator defined on scalars as
$$\begin{matrix}{H}_{λ} ( x ) = \{ \begin{matrix}\frac{2}{3} x ( 1 + cos ( \frac{2 π}{3} - \frac{2 ϕ ( x )}{3} ) ) , & \vert x \vert > \frac{\sqrt[3]{54}}{4} {λ}^{2 / 3} \\ 0 & otherwise\end{matrix} \\ where \\ ϕ ( x ) = {cos}^{- 1} [ \frac{λ}{8} \cdot {( \frac{\vert x \vert}{3} )}^{- 3 / 2} ] .\end{matrix}$$

8. A magnetic resonance imaging (MRI) data-analysis system comprising one or more computing devices configured to execute a process for determining a background component and a dynamic component of an image frame from a sensed data sequence obtained in a dynamic MM application, the sensed data sequence being under-sampled with respect to the image frame, wherein the process is arranged according to the method of claim 1.

9. A magnetic resonance imaging (MRI) data-analysis system comprising one or more computing devices configured to execute a process for determining a background component and a dynamic component of an image frame from a sensed data sequence obtained in a dynamic MM application, the sensed data sequence being under-sampled with respect to the image frame, wherein the process is arranged according to the method of claim 2.

10. A magnetic resonance imaging (MRI) data-analysis system comprising one or more computing devices configured to execute a process for determining a background component and a dynamic component of an image frame from a sensed data sequence obtained in a dynamic MRI application, the sensed data sequence being under-sampled with respect to the image frame, wherein the process is arranged according to the method of claim 3.

11. A magnetic resonance imaging (MRI) data-analysis system comprising one or more computing devices configured to execute a process for determining a background component and a dynamic component of an image frame from a sensed data sequence obtained in a dynamic MRI application, the sensed data sequence being under-sampled with respect to the image frame, wherein the process is arranged according to the method of claim 4.

12. A magnetic resonance imaging (MRI) data-analysis system comprising one or more computing devices configured to execute a process for determining a background component and a dynamic component of an image frame from a sensed data sequence obtained in a dynamic MRI application, the sensed data sequence being under-sampled with respect to the image frame, wherein the process is arranged according to the method of claim 5.

13. A magnetic resonance imaging (MRI) data-analysis system comprising one or more computing devices configured to execute a process for determining a background component and a dynamic component of an image frame from a sensed data sequence obtained in a dynamic MRI application, the sensed data sequence being under-sampled with respect to the image frame, wherein the process is arranged according to the method of claim 6.

14. A magnetic resonance imaging (MRI) data-analysis system comprising one or more computing devices configured to execute a process for determining a background component and a dynamic component of an image frame from a sensed data sequence obtained in a dynamic MRI application, the sensed data sequence being under-sampled with respect to the image frame, wherein the process is arranged according to the method of claim 7.
